(19) United States
(12) Patent Application Publication
(10) Pub. No.: US 2006/0105322 A1
(43) Pub. Date: May 18, 2006

(54) Intracellular metabolic flux analysis method using substrate labeled with isotope

(75) Inventors: Shintaro Iwatani, Kanagawa (JP); Stephen Van Dien, Kanagawa (JP); Yoshihiro Usuda, Kanagawa (JP); Kazuhiko Matsui, Kanagawa (JP)

Correspondence Address: FOLEY AND LARDNER LLP;SUITE 500, 3000 K STREET NW, WASHINGTON, DC, 20007, US

(73) Assignee: AJINOMOTO CO., INC.

(21) Appl. No.: 11/319,104

(22) Filed: Dec. 28, 2005

Related U.S. Application Data
(63) Continuation of application No. PCT/JP04/09602, filed on Jun. 30, 2004.

(30) Foreign Application Priority Data
Jun. 30, 2003 (JP) 2003-187516

(51) Int. Cl. C12Q 1/00; G06F 19/00

(52) U.S. Cl. 435/4; 702/19

(57) ABSTRACT

A method for analyzing an intracellular metabolic flux comprising determining the intracellular metabolic flux from analytical values of cells cultured in a medium containing an isotope-labeled substrate as a carbon source on the basis of an intracellular metabolic flux model constructed for the intracellular metabolic flux to be analyzed, wherein (a) influence of an exchange reaction between an intracellular metabolite and a cell component produced by integration of the intracellular metabolite is considered, (b) uptake of a compound in a medium into cells, which compound is identical to an intracellular metabolite and unlabeled with an isotope, is considered, or (c) carbon dioxide used in a fixation reaction is assumed as carbon dioxide produced in a production reaction.

### TECHNICAL FIELD

The present invention relates to a method for analyzing a metabolic flux, that is, a metabolic flux analysis method, a program for the method and a recording medium recording the program. Specifically, the present invention relates to a metabolic flux analysis method using an isotope-labeled substance, a program for the method and a recording medium recording the program.

### BACKGROUND ART

The metabolic flux analysis method is a method for quantitatively determining an intracellular metabolic flux by analyzing intracellular balances of metabolites or isotope-labeled compounds and conducting isotope compound tracer experiments with an analytical technique such as nuclear magnetic resonance (NMR) or mass spectrometry (MS). In recent years, this method has drawn attentions as a technique for stoichiometrically analyzing the quantitative ratio of metabolites (carbon balance) in metabolic pathways in an objective cell (Non-patent document 1).

Various studies are being conducted to develop an accurate analytical technique for use in metabolic flux analyses. The theory concerning metabolic flux analysis using isotope-labeled substrates has been reported in many papers and is being established (Non-patent documents 2, 3, 4 and 5). Although many experiments are being conducted to establish a metabolic flux analysis method, researches based on a continuous culture method utilizing a synthetic medium as an ideal condition are common to obtain high analytical precision (Non-patent document 6). Further, although there are a few reports on metabolic flux analysis performed by batch culture as a more practical culture method, only isotope distributions of several substances discharged in a medium have been measured, and no calculation has been performed at all on the basis of the measurement of isotope distributions in intracellular substances (Non-patent document 7). Meanwhile, as disclosed in Patent documents 1, 2 and 3, many attempts have been made to theoretically predict a metabolic flux. However, in view of practical use such as applications, these methods are far inferior to the metabolic flux analysis using isotope-labeled substrates (Non-patent documents 8 and 9).
[Non-patent document 1]
Metabolic Engineering, 3, pp. 265-283, 2001
[Non-patent document 2]
Biotechnology and Bioengineering, 55, pp. 101-117, 1997
[Non-patent document 3]
Biotechnology and Bioengineering, 55, pp. 118-135, 1997
[Non-patent document 4]
Biotechnology and Bioengineering, 66, pp. 69-85, 1999
[Non-patent document 5]
Biotechnology and Bioengineering, 66, pp. 86-103, 1999
[Non-patent document 6]
Journal of Biological Chemistry, 275, pp. 35932-35941, 2000
[Non-patent document 7]
European Journal of Biochemistry, 268, pp. 2441-2455, 2001
[Patent document 1]
International Patent Publication No. WO00/46405
[Patent document 2]
International Patent Publication No. WO02/061115
[Patent document 3]
International Patent Publication No. WO02/055995
[Non-patent document 8]
Journal of Biotechnology, 94, pp. 37-63, 2002
[Non-patent document 9]
Metabolic Engineering, 3, pp. 195-205, 2001

### DISCLOSURE OF THE INVENTION

With conventional techniques, it has been difficult to predict accurate metabolic flux distributions reflecting actual states in a culture method or medium used in a usual experiment or actual industrial production. In particular, in metabolic flux analysis using isotope-labeled compounds, errors generated due to contamination with unlabeled substrates must be accepted in current situations. In actual industrial production, nutrients derived from natural raw materials containing nitrogen sources or carbon sources are added to the medium in many cases to increase the initial growth rate, and hence a more precise metabolic flux analysis method in which influence of these unlabeled carbon atoms is corrected is being desired. The present invention provides a metabolic flux analysis method by using isotope-labeled compounds, which exhibits small analytical errors, a method for reducing analytical errors in the metabolic flux analysis using isotope-labeled compounds, a program for executing the aforementioned methods and a recording medium storing the aforementioned program.

The inventors of the present invention assiduously studied considering the aforementioned problems. As a result, they found a method for reducing analytical errors in metabolic flux analysis using isotope-labeled compounds. That is, they have found that, in a method of determining an intracellular metabolic flux from analytical values of cells cultured in a medium containing an isotope-labeled substrate as a carbon source on the basis of an intracellular metabolic flux model constructed for an intracellular metabolic flux to be analyzed, a particular correction or assumption is effective for reducing analytical errors.

Specifically, they have found that the analytical errors can be reduced by making correction for influence of unlabeled compounds in consideration of an exchange reaction occurring between cellular proteins and a intracellular amino acid pool.

They have also found that analytical precision can be improved by constructing a calculation equation considering uptake and decomposition pathways of unlabeled compounds added for the purpose of improvement of growth etc. to take into account the influence of those unlabeled compounds on isotope distributions in various intracellular substances.

Further, to calculate the carbon balance, uptake of carbon dioxide was examined, which is a major carbon source other than isotope-labeled substrates. As a result, they have found that since the concentration of carbon dioxide produced by cells as a result of consumption of isotope-labeled substrates is very high, and thus the carbon balance can be calculated by assuming that the total carbon dioxide partial pressure in a culture broth is attributable to carbon dioxide discharged from the cells.

They have further found that it is effective to make a correction for uptake of compounds comprising unlabeled carbon atoms added to the medium into cellular proteins, when a metabolic flux is calculated by using analytical values of isotope distributions in cellular protein-hydrolyzed amino acids.

The present invention was accomplished on the basis of the aforementioned findings and provides the following:
(1) A method for analyzing an intracellular metabolic flux comprising determining the intracellular metabolic flux from analytical values of cells cultured in a medium containing an isotope-labeled substrate as a carbon source on the basis of an intracellular metabolic flux model constructed for the intracellular metabolic flux to be analyzed,

which satisfies at least one of the following conditions (a) to (c):
(a) the analytical values of cells include an analytical value of isotope distribution in an intracellular metabolite included in the intracellular metabolic flux model, and the analytical value of isotope distribution in the intracellular metabolite is corrected for a degree of synthesis and degradation between the intracellular metabolite and a cell component produced by integration of the intracellular metabolite;
(b) the intracellular metabolic flux model includes at least one of useful compounds and major metabolic intermediates thereof; the analytical values of cells include an uptake rate of a compound in a medium into cells, the compound being identical to the intracellular metabolite and unlabeled with an isotope, and an analytical value of isotope distribution in at least one of the useful compounds and the major metabolic intermediates thereof; and the analytical value of isotope distribution in at least one of the useful compounds and the major metabolic intermediates thereof is corrected for influence of a rate of inflow into a metabolic pathway on the isotope distribution in at least one of the useful compounds and the major metabolic intermediates thereof on the assumption that a rate obtained by subtracting a rate of integration into a cell component from the uptake rate is the rate of inflow into the metabolic pathway;
(c) the intracellular metabolic flux model includes a carbon dioxide fixation reaction and a carbon dioxide production reaction, and carbon dioxide used in the fixation reaction is assumed as carbon dioxide produced in the production reaction.
(2) The method according to (1), which satisfies the condition (a), and wherein the analytical value of the isotope distribution in the intracellular metabolite is corrected by constructing the intracellular metabolic flux model to include an exchange reaction of the intracellular metabolite and the cell component produced by integration of the intracellular metabolite, and using the analytical values of cells including the analytical value of the isotope distribution in the intracellular metabolite and an analytical value of isotope distribution in a degradation product of the cell component.
(3) The method according to (1), which satisfies the condition (a), and wherein the analytical value of isotope distribution in the intracellular metabolite is corrected by 1) the step of measuring isotope distribution in the intracellular metabolite and isotope distribution in a degradation product of the cell component, and 2) the step of optimizing the degree of synthesis and degradation between the intracellular metabolite and the cell component on the basis of the results obtained in the step 1), by an optimization algorithm.
(4) The method according to (3), wherein the degree of synthesis and degradation is expressed as a variable defined by an exchange reaction coefficient.
(5) The method according to (3) or (4), wherein the optimization algorithm is an evolutionary algorithm.
(6) The method according to any one of (3) to (5), wherein the intracellular metabolite is at least one of an amino acid and an organic acid, and the cell component is a protein.
(7) The method according to any one of (2) to (6), which satisfies the condition (a), and wherein the analytical value of the isotope distribution in the degradation product of the cell component is corrected for influence of integration of a compound in the medium into the cell component, the compound being identical to the intracellular metabolite and unlabeled with an isotope.
(8) The method according to (7), wherein the compound which is unlabeled with an isotope is an amino acid.
(9) The method according to (1), which satisfies the condition (b), and wherein the compound which is unlabeled with an isotope is an amino acid.
(10) The method according to (9), wherein the amino acid is isoleucine.
(11) The method according to any one of (1) to (10), wherein the cells are those of a microorganism having an ability to produce a useful compound.
(12) The method according to (11), wherein the useful compound is at least one of an amino acid and an organic acid.
(13) The method according to any one of (1) to (12), wherein culture of the cells is batch culture or fed-batch culture.
(14) The method according to any one of (1) to (13), wherein the intracellular metabolite is at least one of an amino acid and an organic acid, or a major metabolic intermediate thereof, or both.
(15) The method according to any one of (1) to (14), wherein the isotope distribution is measured by mass spectrometry.
(16) A program for causing a computer to function as a means for storing an intracellular metabolic flux model constructed for an intracellular metabolic flux to be analyzed, a means for inputting analytical values of cells cultured in a medium containing isotope-labeled substrates as a carbon source, a means for determining a variable of the intracellular metabolic flux model on the basis of the intracellular metabolic flux model and the analytical values of cells to determine the intracellular metabolic flux and a means for outputting the determined intracellular metabolic flux, wherein the intracellular metabolic flux model is constructed, or the variable of the intracellular metabolic flux model is calculated, or both, so that at least one of the following conditions (a) to (c) is satisfied:
(a) the analytical values of cells include an analytical value of isotope distribution in an intracellular metabolite included in the intracellular metabolic flux model, and the analytical value of isotope distribution in the intracellular metabolite is corrected for a degree of synthesis and degradation between the intracellular metabolite and a cell component produced by integration of the intracellular metabolite;
(b) the intracellular metabolic flux model includes at least one of useful compounds and major metabolic intermediates thereof; the analytical values of cells include an uptake rate of a compound in a medium into cells, the compound being identical to the intracellular metabolite and unlabeled with an isotope, and an analytical value of isotope distribution in at least one of the useful compounds and the major metabolic intermediates thereof; and the analytical value of isotope distribution in at least one of the useful compounds and the major metabolic intermediates thereof are corrected for influence of a rate of inflow into a metabolic pathway on the isotope distribution in at least one of the useful compounds and the major metabolic intermediates thereof on the assumption that a rate obtained by subtracting a rate of integration into a cell component from the uptake rate should be the rate of inflow into the metabolic pathway;
(c) the intracellular metabolic flux model includes a carbon dioxide fixation reaction and a carbon dioxide production reaction, and carbon dioxide used in the fixation reaction is assumed as carbon dioxide produced in the production reaction.
(17) A computer-readable recording medium, which records the program as defined in (16).

### BRIEF DESCRIPTION OF THE DRAWINGS

FIG. 1 shows relationship between uptake of unlabeled amino acids derived from a medium and exchange reactions of intracellular proteins and intracellular amino acid pools. In the initial stage of cultivation (cultivation for about 12 hours), the unlabeled amino acids derived from the medium were being taken up. During the growth phase (at about 17 hours after the start of cultivation), all unlabeled amino acids derived from yeast extract were consumed, whereas isoleucine, a growth promoting factor, remained in the medium. Its consumption rate V_{Ile} was measured, and it was assumed that it was due to decomposition by metabolism. V_{YE} represents an uptake flux of amino acids from the medium into a bacterium. Pex represents an exchange reaction coefficient of intracellular proteins and intracellular amino acid pools. Pex is a variable determined by an optimization algorithm.

FIG. 2 shows analytical values of the culture including absorbance (OD), specific growth rate μ, specific sugar consumption rate ν, specific lysine production rate σ, oxygen absorption rate rab and respiratory quotient RQ of cells.

FIGS. 3A and 3B show concentrations of amino acids and acetic acid in the medium: Asp: aspartic acid, Thr: threonine, Ser: serine, Leu: leucine, Gly: glycine, Ala: alanine, Cys: cysteine, Val: valine, Met: methionine, Tyr: tyrosine, Phe: phenylalanine, His: histidine, Arg: arginine, Glu: glutamic acid, Ile: isoleucine, Lys(Base): lysine and AcOH: acetic acid.

FIG. 4 shows a metabolic flux distribution (growth phase, at 17 hours after the start of cultivation) calculated from measured values of the isotope distribution in protein-hydrolyzed amino acids. Each numerical value represents change in amount of each substance in a unit of mmol with respect to 10 mmol of glucose.

FIG. 5 shows a metabolic flux distribution (stationary phase, at 26 hours after the start of cultivation) calculated from measured values of the isotope distribution in protein-hydrolyzed amino acids. Each numerical value represents change in amount of each substance in a unit of mmol with respect to 10 mmol of glucose.

FIG. 6 shows a metabolic flux distribution (growth phase, at 17 hours after the start of cultivation) calculated from measured values of the isotope distribution in intracellular amino acids. Each numerical value represents change in amount of each substance in a unit of mmol with respect to 10 mmol of glucose.

FIG. 7 shows a metabolic flux distribution (stationary phase, at 26 hours after the start of cultivation) calculated from measured values of the isotope distribution in intracellular amino acids. Each numerical value represents change in amount of each substance in a unit of mmol with respect to 10 mmol of glucose.

FIG. 8 is a flowchart of a program for analysis of an intracellular metabolic flux.

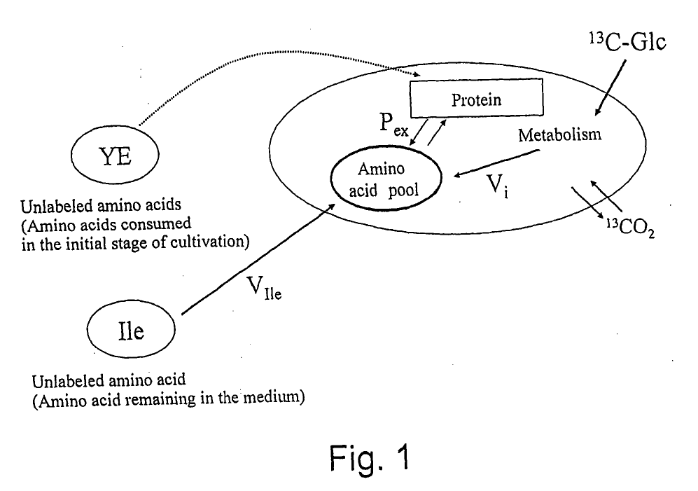

### BEST MODE FOR CARRYING OUT THE INVENTION

Hereafter, the present invention will be explained in detail.

The intracellular metabolic flux referred to in the present invention is a flux of an intracellular metabolite derived from a stoichiometric model of an intracellular chemical reaction and the law of mass action between metabolites.

The intracellular metabolite referred to in the present invention is a substance metabolized in a cell. Many findings about intracellular metabolites as well as the biochemical reactions thereof have been obtained and accumulated in databases (refer to, for example, Kyoto Encyclopedia of Genes and Genomes (KEGG, http://www.genome.ad.jp/kegg/).

The cell component referred to in the present invention is a substance constituting a cell, which is produced by integration of the intracellular metabolite. Examples thereof include substances such as proteins, carbohydrates, nucleic acids and lipids. Further, a degradation product of the cell component means a degradation product at the same level of the intracellular metabolites integrated into the cell component. For example, when the cell component is a protein produced by integration of amino acids, the degradation product is an amino acid. In the present specification, when the cell component is a protein produced by integration of amino acids, in particular, it is also referred to as a cellular protein. Further, an amino acid as a degradation product of a cellular protein is also referred to as a cellular protein-hydrolyzed amino acid.

Any cell can be the cell analyzed in the present invention, and examples thereof include, in particular, cells used for production of a substance, such as various cultured cells, fungi, yeasts and various bacteria. They are preferably microorganisms having an ability to produce useful compounds, for example, amino acids, nucleic acids or organic acids. Preferred examples of the microorganisms having an ability to produce amino acids, nucleic acids or organic acids include Escherichia coli, Bacillus bacteria, coryneform bacteria and so forth.

The isotope used in the present invention is usually a stable isotope. However, radioactive isotopes can also be used for the same purpose. Examples of isotope-labeled substrates include isotope-labeled glucose, specifically, glucose having a carbon atom labeled with a stable isotope at the 1-position and/or glucose having all of which carbon atoms are labeled with stable isotopes. An example of the isotope is ¹³C.

The intracellular metabolic flux model used in the present invention is not particularly limited so long as it is constructed for a metabolic flux to be analyzed, and an intracellular metabolic flux model constructed according to a usual construction method is sufficient. The expression “constructed for a metabolic flux” means that a reaction (reaction pathway) for a metabolic flux to be analyzed is included in the constructed intracellular metabolic flux model.

Examples of the method for constructing an intracellular metabolic flux model for a metabolic flux include the methods described in Metabolic Engineering, 3, pp. 265-283, 2001 (Non-patent document 1); Wiechert, W. and de Graaf, A. A., Biotechnology and Bioengineering, 55, pp. 101-117, 1997 (Non-patent document 2); Metabolic Engineering, 3, pp. 195-205, 2001 (Non-patent document 9), Metabolic Engineering, 3, pp. 173-191, 2001; Biotechnology and Bioengineering, 55, pp. 831-840 and so forth.

The reaction pathway used for the analysis of a metabolic flux may be any reaction pathway so long as it is a major intracellular metabolic pathway, and in particular, glycolysis pathways, TCA cycle, pentose phosphate pathway and pathways specific to various amino acid syntheses are preferably included because they are important in practical production of useful compounds by microbial fermentation.

In the construction of an intracellular metabolic flux model, reaction pathways may be simplified by assuming a series of reactions with no branching as one reaction, assuming metabolites converted by a reaction of a high metabolic rate before and after the reaction as one metabolite and so forth.

The expression “analytical values of cells” means measurable analytical values concerning cells cultured in a medium containing an isotope-labeled substrate as a carbon source, and examples thereof include analytical values of isotope distributions in metabolites, bacterial cell production rate, useful substance production rate and so forth. The analytical values of isotope distributions are not particularly limited so long as they reflect isotope distributions, and examples thereof include isotopomer distribution vectors (Biotechnology and Bioengineering, 55, pp. 831-840), mass distribution vectors (Biotechnology and Bioengineering, 62, pp. 739-750) and so forth. Because measurement by mass spectrometry is possible, mass distribution vectors are preferred.

The step of determining an intracellular metabolic flux from analytical values of cells cultured in a medium containing an isotope-labeled substrate as a carbon source can be performed according to a usual determination method. When the analytical values include analytical values of isotope distributions, the determination is usually made by using an isotopomer balance equation (refer to, for example, Biotechnology and Bioengineering, 66, pp. 69-85, 1999 (Non-patent document 4)).

When the analytical values of cells are sufficient to calculate variables in a metabolic flux model (when the metabolic flux model is represented by a stoichiometric matrix, a solution is obtained), variables in the metabolic flux model are determined on the basis of the analytical values of cells, and thereby the metabolic flux can be determined. When the analytical values of cells are not sufficient to calculate variables in the metabolic flux model, part of variables other than isotope distribution in the metabolic flux model is/are usually used as free variable(s), and on the basis of the free variable(s), the analytical values of cells other than the analytical value of the isotope distribution and the labeling pattern in the used substrate (positions and number of isotopes, and proportions of substrates when two or more kinds of substrates having different number of isotopes at different positions are used), optimization is performed by comparison between the value of the isotope distribution calculated from the metabolic flux model and the analytical value of the isotope distribution to determine variables in the metabolic flux model. Thus, the metabolic flux can be determined. Examples of such an optimization method include the methods described in Metabolic Engineering, 3, pp. 265-283, 2001 (Non-patent document 1), Biotechnology and Bioengineering, 55, pp. 118-135, 1997 (Non-patent document 3), Biotechnology and Bioengineering, 66, pp. 69-85, 1999 (Non-patent document 4) and so forth.

In the step of determining an intracellular metabolic flux from the analytical value of the isotope distribution in the metabolite in cells cultured in a medium containing an isotope-labeled substrate as a carbon source, the labeling pattern of the substrate can be determined by a usual method (refer to, for example, Biotechnology and Bioengineering, 66, pp. 86-103, 1999 (Non-patent document 5), European Journal of Biochemistry, 268, pp. 2441-2455, 2001 (Non-patent document 7)).

The method of the present invention is a method for analyzing an intracellular metabolic flux from analytical values of cells cultured in a medium containing an isotope-labeled substrate as a carbon source on the basis of an intracellular metabolic flux model constructed for the intracellular metabolic flux to be analyzed, which is characterized in that it satisfies at least one of the following conditions (a) to (c).
(a) The analytical values of cells include an analytical value of isotope distribution in an intracellular metabolite included in the intracellular metabolic flux model, and the analytical value of isotope distribution in the intracellular metabolite is corrected for a degree of synthesis and degradation between the intracellular metabolite and a cell component produced by integration of the intracellular metabolite.
(b) The intracellular metabolic flux model includes a useful compound and/or a major metabolic intermediate thereof; the analytical values of cells include an uptake rate of a compound in a medium into cells, which compound is identical to the intracellular metabolite and unlabeled with an isotope, and an analytical value or values of isotope distribution in the useful compound and/or the major metabolic intermediate thereof; and the analytical value or values of isotope distribution in the useful compound and/or the major metabolic intermediate thereof are corrected for influence of a rate of inflow into a metabolic pathway on the isotope distribution in the useful compound and/or the major metabolic intermediate thereof on the assumption that a rate obtained by subtracting a rate of integration into a cell component from the uptake rate is the rate of inflow into the metabolic pathway.
(c) The intracellular metabolic flux model includes a carbon dioxide fixation reaction and a carbon dioxide production reaction, and carbon dioxide used in the fixation reaction is assumed as carbon dioxide produced in the production reaction.

Each condition will be explained below.

According to the condition (a), in calculation of the metabolic flux, the isotope distribution in the intracellular metabolite (e.g. amino acid) is corrected in consideration of the influence of an intracellular metabolite produced by degradation of an intracellular component (e.g. cellular protein) produced in the cell growth phase, that is, an exchange reaction between intracellular metabolite pool and intracellular metabolites produced by degradation of the intracellular component. As for the correction method, the analytical value of the isotope distribution in the intracellular metabolite may be corrected on the basis of the exchange reaction, or the exchange reaction may be included in the intracellular metabolic flux model. When the exchange reaction is included in the intracellular metabolic flux model, the intracellular metabolic flux model includes the exchange reaction between the intracellular metabolite and the cell component produced by integration of the intracellular metabolite, and the analytical values of cells include the analytical value of the isotope distribution in the intracellular metabolite and an analytical value of isotope distribution in the degradation product of cell component. When the exchange reaction is included in the intracellular metabolic flux model, a corrected analytical value of the isotope distribution in the intracellular metabolite is not directly used. However, by determining the intracellular metabolic flux on the basis of the metabolic flux model, the analytical value of the isotope distribution in intracellular metabolite is become to be corrected as a result.

Specific examples of the method for correcting isotope distribution in an intracellular metabolite include a method of constructing the intracellular metabolic flux model to include an exchange reaction between the intracellular metabolite and the cell component produced by integration of the intracellular metabolite so that the analytical values of the isotope distributions in the intracellular metabolite and the degradation product of the cell component are included in the analytical values of cells.

Another example of the correction method is a method comprising 1) the step of measuring isotope distribution in the intracellular metabolite and isotope distribution in a degradation product of the cell component, and 2) the step of optimizing the degree of synthesis and degradation between the intracellular metabolite and the cell component on the basis of the results obtained in the step 1) by an optimization algorithm. In this embodiment, the degree of synthesis and degradation is preferably expressed by using a variable defined with an exchange reaction coefficient. Further, examples of the optimization method include the evolutionary algorithm (Journal of Theoretical Biology, 199, pp. 45-61, 1999) and other methods, and the evolutionary algorithm is preferred. In this embodiment, it is preferred that the intracellular metabolite is an amino acid and/or an organic acid, and that the cell component is a protein.

In an embodiment using the analytical value of isotope distribution in the degradation product of the cell component, the analytical value of isotope distribution in the degradation product of the cell component is preferably corrected in consideration of the influence of integration of a compound in the medium into the cell component, which compound is identical to the intracellular metabolite and unlabeled with an isotope. For example, when the cell component is a protein, the integration of an amino acid unlabeled with an isotope in the medium into the cellular protein is corrected when a metabolic flux is calculated by using analytical values of isotope distributions in cellular protein-hydrolyzed amino acids.

According to the condition (b), when a compound which is identical to an intracellular metabolite and unlabeled with an isotope is contained in the medium, the rate at which it is taken up into cells is analyzed. Then, the influence on the isotope distribution in an intracellular useful compound and/or a major metabolic intermediate thereof is corrected on the assumption that the rate obtained by subtracting the rate used for a cell component from the uptake rate is a flux for a flow into the decomposition pathway. As for the correction method, the analytical value of the isotope distribution in the intracellular metabolite may be corrected on the basis of the flux for the flow into the decomposition pathway, or the aforementioned flow rate may be included in the intracellular metabolic flux model. In this embodiment, the compound that is not labeled with an isotope is preferably an amino acid (preferably isoleucine).

The term “useful compound” used herein means compounds useful for seasoning, feed additives and pharmaceuticals, such as, amino acids, organic acids and nucleic acids.

The term “major metabolic intermediate” used herein means all metabolic intermediates included in metabolic flux analysis model, such as pyruvate, glucose-6-phosphate, fructose-6-phosphate, oxaloacetate, and so on.

According to the condition (c), the carbon balance is calculated on the assumption that the total carbon dioxide partial pressure in a culture broth is attributable to carbon dioxide discharged from cells as a result of consumption of the isotope-labeled substrate.

In the present invention, correction or assumption is performed so that any one of the aforementioned conditions is satisfied. This can reduce analytical errors in the metabolic flux analysis using the isotope-labeled compound.

In the present invention, the cells are preferably those of a microorganism having an ability to produce a useful compound. Examples of the cells include those of Escherichia coli, coryneform bacteria and Bacillus bacteria. The useful compound is preferably an amino acid and/or an organic acid.

In the present invention, culture of the cells is preferably batch culture or fed-batch culture. The batch culture is a closed system culture method with specific nutrient types, whereas the fed-batch culture is a culture method in which a substrate is continuously or intermittently added to a feeding medium in the culture system. In the analysis method of the present invention, the effect of reducing analytical errors becomes more significant when the culture is performed as batch culture or fed-batch culture.

In the present invention, the intracellular metabolite is preferably an amino acid and/or organic acid and/or major metabolic intermediate thereof.

In the present invention, the isotope distribution is preferably measured by mass spectrometry.

The present invention also provides a program for executing the analysis method of the present invention. The program of the present invention is a program for causing a computer to function as a means for storing an intracellular metabolic flux model constructed for an intracellular metabolic flux to be analyzed, a means for inputting analytical values of cells cultured in a medium containing isotope-labeled substrates as a carbon source, a means for determining a variable of the intracellular metabolic flux model on the basis of the intracellular metabolic flux model and the analytical values of cells to determine the intracellular metabolic flux and a means for outputting the determined intracellular metabolic flux, wherein the intracellular metabolic flux model is constructed, and/or the variable of the intracellular metabolic flux model is calculated so that at least one of the aforementioned conditions (a) to (c) is satisfied.

Further, another embodiment of the present invention relates to a computer-readable recording medium, in which the aforementioned program is recorded.

The intracellular metabolic flux model constructed for the intracellular metabolic flux to be analyzed and the analytical values of cells cultured in a medium containing an isotope-labeled substrate as a carbon source are as explained for the analysis method of the present invention. The intracellular metabolic flux model is usually stored in a format of data usually used for representation of an intracellular metabolic flux model. For example, when the metabolic flux model is represented by a stoichiometric matrix, the model data are stored as a matrix. The means for inputting analytical values include a means for transmitting data from a storage medium or via a transmission medium.

The means for determining a variable of the intracellular metabolic flux model on the basis of the intracellular metabolic flux model and analytical values of cells to determine the intracellular metabolic flux may be a means suitable for performing the determination step explained in the analysis method of the present invention.

The means for outputting the determined intracellular metabolic flux includes a means for transferring data to the storage medium or via the transmission medium. The output of the intracellular metabolic flux may be a chart showing a metabolic network for which the metabolic flux model is constructed and displaying flux values at positions corresponding to respective reactions in the metabolic network in the chart.

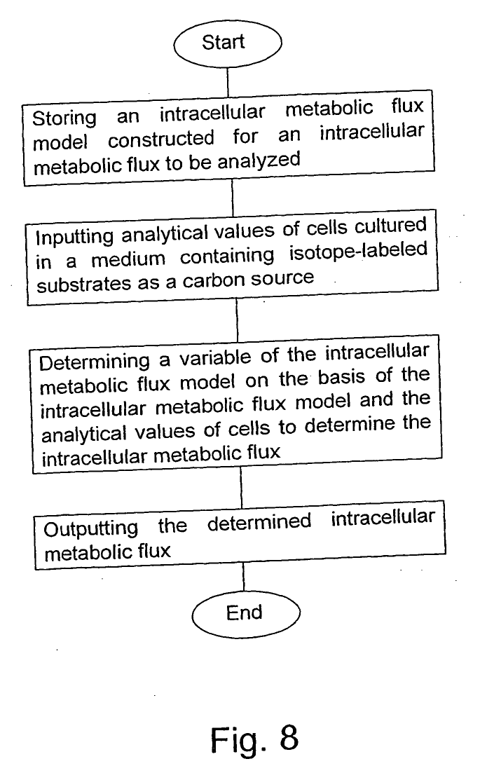

The flowchart of the program of the present invention is shown in FIG. 8. The aforementioned conditions (a) to (c) and preferred embodiments thereof are as explained for the analysis method of the present invention, and the program of the present invention can be prepared according to a usual programming method except that the intracellular metabolic flux model is constructed, and/or the variable of the intracellular metabolic flux model is calculated so that the aforementioned conditions are satisfied.

The recording medium in which the program of the present invention is recorded includes any of removable physical media such as a flexible disk, a magneto-optical, ROM, EPROM, EEPROM, CD-ROM, DVD and the like; any of fixed physical media built in various computer systems such as ROM, RAM, HD and the like; and any of communication media in which the program is stored in a short term such as communication circuits and carrier wave in the case of transmission of programs via a network represented by LAN, WAN and the Internet.

### EXAMPLES

The bacterial strains and media shown below were used.

(1) Escherichia coli Strain and Plasmid

Bacterial strain: WYK050 (a strain derived from Escherichia coli wild strain W3110, which is resistant to S-(2-aminoethyl)cysteine and deficient in lysine decomposition genes, ldc and cadA genes (Kikuchi, Y. et al. J. Bacteriol., 179, pp. 4486-4492, 1997))
Plasmid: pCAB1 (obtained by incorporating lysC, dapA and dapB genes derived from Escherichia coli into vector RSF1010)

A bacterial strain obtained by introducing pCAB1 into WYK050 was used for cultivation.

(2) Media

LB agar medium: 1.0% Bacto tryptone, 0.5% Bacto yeast extract, 1% NaCl, 1.5% agar. If necessary, 20 μg/ml of streptomycin was added.
Main culture medium: 16 g/L of ammonium sulfate, 3 g/L of potassium dihydrogenphosphate, 4 g/L of yeast extract, 10 mg/L of iron sulfate heptahydrate, 10 mg/L of manganese sulfate pentahydrate, 400 mg/L of isoleucine, 40 g/L of glucose, 1 g/L of magnesium sulfate heptahydrate. pH was adjusted to 7.0 with potassium hydroxide. If necessary, 20 μg/ml of streptomycin was added. The main culture medium was used for liquid culture of Escherichia coli.
Feeding solution: 500 g/L of glucose, 80 g/L of ammonium sulfate

### Example 1

(1) Construction of Metabolic Flux Analysis Model

A stoichiometric equation for calculating a metabolic flux was developed by assuming a quasi-steady state of intracellular metabolic intermediates (Savinell and Palsson, Journal of Theoretical Biology, 154, pp. 421-454, 1992; Vallino and Stephanopoulos, Biotechnology and Bioengineering, 41, pp. 633-646, 1993). Formulas of the reactions included in this model are as shown in Table 2. Explanations of the abbreviations are given in Table 1. Some reactions without branching were consolidated to simplify the formula. Since the pentose phosphate pathway is complicated, it was represented by using two formulas. For biomass composition, previously reported data was used (Neidhardt et al., Physiology of the Bacterial Cell, 1990). Further, the composition of amino acids in intracellular proteins was obtained from the concentration ratios of the amino acids obtained by actually hydrolyzing the intracellular proteins. The stoichiometric matrix of this model has a degree of freedom of 8, and 7 fluxes other than the sugar consumption rate must be determined to obtain a solution. The following 7 fluxes were defined as the free fluxes: bacterial cell production rate, lysine production rate, acetic acid production rate, formic acid production rate, ICL flux, G6PDH flux and malic enzyme flux. The results of the cell production rate and various production rates were obtained from the cultivation experiment. Further, the remaining 3 fluxes were determined by an optimization algorithm on the basis of measured values of the isotope distributions in amino acids and so forth (described later). Further, the constructed model includes 14 reversible reactions. Their reversibilities were defined as exchange coefficients that can be represented by numerical values of 0 to 1 (Dauner et al., Biotechnology and Bioengineering, 76, pp. 144-156, 2001; Wiechert and de Graaf, Biotechnology and Bioengineering, 55, pp. 101-117, 1997). These exchange coefficients are also variables determined on the basis of the measured values of the isotope distributions as the aforementioned 3 free fluxes. As for neighboring reactions in the glycolysis, pentose phosphate pathway and TCA cycle, the reversibilities were assumed to be equal for simplification. Since the results of sensitivity analysis revealed that the reactions 9, 29 and 30 in the reaction list of Table 2 had little influence on the isotope distributions, the values were assumed to be 0. From the above, reversible reactions of which exchange coefficients were to be determined were 6 reactions.

To calculate isotopomer distribution vectors (IDV) of all the substances in the model, an isotopomer balance equation was developed as a function of free fluxes and exchange coefficients and isotopomer distributions in substrates. A column vector called IDV represents proportions of isotopomers, and the sum of elements is 1 (Schmidt et al., Biotechnology and Bioengineering, 55, pp. 831-840, 1997; Wittmann and Heinzle, Biotechnology and Bioengineering, 62, pp. 739-750, 1999). The isotopomer balance equation is described by using an isotopomer mapping matrix (IMM) explained in more detail by Schmidt et al. (Schmidt et al., Biotechnology and Bioengineering, 55, pp. 831-840, 1997). An atom mapping matrix (AMM) is a matrix representing transfer of carbon atoms from a reactant to a product. On the basis of this, the isotopomer mapping matrix (IMM), which represents transfer of isotopomers from a reactant to a product, is computed by using MATLAB (The MathWorks, Natick, Mass.), which is a mathematical software.

The isotopomer balance equation can be solved by using the Gause-Seidel iteration method with the free fluxes and exchange coefficients as inputs.

In addition to consumption of glucose, a microbial cell takes up carbon dioxide and consumes acetic acid during the growth. Since carbon dioxide is also produced from metabolism of isotope-labeled glucose, some percentages of carbon dioxide consist of ¹³C-carbon dioxide. The percentage was calculated according to a carbon dioxide balance equation taking all the reactions producing carbon dioxide into consideration. Although accurate value varies depending on the intracellular metabolic flux distribution, it was generally about 32%. In this calculation, it was assumed that carbon dioxide from air was not consumed. This is because the concentration of carbon dioxide produced by the cells as a result of consumption of isotope-labeled glucose is very high (in the experiment, the concentration of exhausted carbon dioxide reached 4 to 5%), and therefore it may be considered that the total carbon dioxide partial pressure in a fermenter should be attributable to carbon dioxide exhausted from the cells.

Although isotopomer distributions cannot be obtained for all of the substances from the mass spectrometry analysis, mass distributions can be obtained. This information is represented as mass distribution vector (MDV), and each element includes an isotopomer having an identical mass (Wittman and Heinzle, Biotechnology and Bioengineering, 62, pp. 739-750, 1999). Therefore, for a substance having n of carbon atoms, MDV contains n+1 of elements. MDV can be calculated by adding up elements having an identical mass among those in IDV. To what degree the result of the model matches the experimental value can be evaluated by comparing the MDV calculated as described above with the MDV obtained from the experiment.

TABLE 1

| | |
|---|---|
| μ | Specific growth rate [h⁻¹] |
| ν | Specific sugar consumption rate [g/g/h] |
| ρ | Specific lysine production rate [g/g/h] |
| YE | Yeast extract |
| ldc | E. coli lysine decarboxylase gene (Constitutive) |
| cadA | E. coli lysine decarboxylase gene (Inducible) |
| lysC | E. coli aspartate kinase III gene |
| dapA | E. coli dihydrodipicolinate synthase gene |
| dapB | E. coli dihydrodipicolinate reductase gene |
| CT | Cultivation time |
| ICL | Isocitrate lyase |
| PP pathway | Pentose phosphate pathway |
| PEPC | Phosphoenolpyruvate carboxylase |
| ICD | Isocitrate dehydrogenase |
| DDH | meso-Diaminopimelate dehydrogenase |
| G6PDH | Glucose-6-phosphate dehydrogenase |
| 3PG | 3-Phospho-D-glyceric acid |
| AcCoA | Acetyl coenzyme A |
| AcOH | Acetic acid |
| aIVA | α-Keto-isovaleric acid |
| aKG | 2-Oxoglutaric acid |
| Ala | Alanine |
| Arg | Arginine |
| Asn | Asparagine |
| Asp | Aspartic acid |
| CHR | Chorismic acid |
| Cit | Citric acid |
| CO2 | Carbon dioxide |
| Cys | Cysteine |
| E4P | Erythrose-4-phosphate |
| extraC1 | Carbon atom derived from ATP curing histidine |
| | synthesis |
| F6P | Fructose-6-phosphate |
| Form | Formic acid |
| Fum | Fumaric acid |
| G6P | Glucose-6-phosphate |
| GAP | Glyceraldehyde-3-phosphate |
| Glc | Glucose |
| Gln | Glutamine |
| Glu | Glutamic acid |
| Gly | Glycine |
| His | Histidine |
| Ile | Isoleucine |
| Leu | Leucine |
| Lys | Lysine |
| Lysext | Lysine product (secreted) |
| Mal | Malic acid |
| Met | Methionine |
| mTHF | Methyltetrahydrofolic acid |
| NH3 | Ammonia |
| OAA | Oxaloacetatic acid |
| PEP | Phosphoenolpyruvic acid |
| Phe | Phenylalanine |
| Pro | Proline |
| PRPP | Phosphoribosyl pyrophosphate |
| Pyr | Pyruvic acid |
| R5P | Pentose phosphate pool |
| SDAP | N-Succinyl-L-2,6-diaminoheptanedioate |
| Ser | Serine |
| Suc | Succinic acid |
| THF | Tetrahydrofolic acid |
| Thr | Threonine |
| Trp | Tryptophan |
| Tyr | Tyrosine |
| Val | Valine |

TABLE 2
Reaction formulas used for metabolic model

| | | |
|---|---|---|
| [1] | | Glc + PEP -> G6P + Pyr |
| [2] | | G6P -> R5P + CO2 |
| [3] | (r) | 3R5P -> 2F6P + GAP |
| [4] | (r) | 2R5P -> F6P + E4P |
| [5] | (r) | G6P -> F6P |
| [6] | (r) | F6P -> 2GAP |
| [7] | (r) | GAP -> 3PG |
| [8] | (r) | 3PG -> PEP |
| [9] | (r) | PEP -> Pyr |
| [10] | | Pyr + CoA -> AcCoA + CO2 |
| [11] | (r) | PEP + CO2 -> OAA |
| [12] | | AcCoA -> AcOH + CoA |
| [13] | | AcCoA + OAA -> Cit + CoA |
| [14] | (r) | Cit -> aKG + CO2 |
| [15] | | aKG + NH₃ -> Glu |
| [16] | | aKG -> Suc + CO2 |
| [17] | | Cit + AcCoA -> Mal + Suc + CO2 + CoA |
| [18] | (r) | Succ -> Mal |
| [19] | (r) | Mal -> OAA |
| [20] | | OAA + Glu -> Asp + aKG |
| [21] | | Asp + Pyr -> Lys + CO2 |
| [22] | | Asp + Pyr + Glu -> Lys + aKG + CO2 |
| [23] | | Glu + NH3 -> Gln |
| [24] | | Glu -> Pro |
| [25] | | Glu + Gln + Asp + AcCoA + CO2 -> Arg + aKG + |
| | | Fum + CoA |
| [26] | | Asp + Cys + mTHF -> Met + CoA + THF + Pyr + NH3 |
| [27] | | Asp -> Thr |
| [28] | | Thr + Glu + Pyr -> Ile + aKG + NH3 + CO2 |
| [29] | (r) | 3PG -> Ser |
| [30] | (r) | Ser + THF -> Gly + mTHF |
| [31] | | 2PEP + E4P -> CHR |
| [32] | | CHR + Glu -> Tyr + CO2 + aKG |
| [33] | | CHR + Glu -> Phe + CO2 + aKG |
| [34] | | CHR + R5P + Ser + Gln -> Trp + Glu + Pyr + CO2 + GAP |
| [35] | | 2Pyr -> aIVA + CO2 |
| [36] | | aIVA + Glu -> Val + aKG |
| [37] | | Val + Pyr -> Ala + aIVA |
| [38] | | aIVA + AcCoA + Glu -> Leu + CO2 + aKG + CoA |
| [39] | | PRPP + Gln + extraC1 -> His + aKG |
| [40] | | Ser + AcCOA + H2S -> Cys + AcOH |
| [41] | | Asp + NH3 -> Asn |
| [42] | (r) | Mal -> Pyr + CO2 |
| [43] | | R5P -> PRPP |
| [44] | | mTHF -> Form |
| [45] | | Gly -> CO2 + mTHF |
| [46] | | Ile + CO2 -> Thr + Pyr |

(r): Reversible reaction

(2) Correction for Naturally Occurring Isotopes of Atoms of Carbon, Hydrogen, Nitrogen and Oxygen

The MDV obtained from the experiment was calculated after corrections were made for naturally occurring ²H (0.01%), ¹⁵N (0.37%), ¹⁷O (0.04%) and ¹⁸O (0.20%). The formula is as follows:
I_{obs}=I_{corr}^{M}+(0.0001N_{H}+0.0037N_{N}+0.0004Nₒ)·I_{corr}^{M-1}+0.002N_{O}·I_{corr}^{M-2}−(0.0001N_{H}+0.0024N_{O}+0.0037N_{N})·I_{corr}^{M}

The corrections for natural isotopes of carbon are not included in the above formula because they were incorporated into IDV of glucose to be used as an input value.

(3) Correction for Unlabeled Amino Acids Contained n Initial Medium

In industrial production, naturally derived nutrients including nitrogen sources and carbon sources are added to a medium to increase the initial growth rate. When MDV calculated from the model and MDV obtained from the experiment are compared, the influence of unlabeled carbon atoms derived from natural components needs to be corrected. Different correction methods were used for analysis of the intracellular amino acids (free amino acids) and for analysis of the protein-hydrolyzed amino acids. It was assumed that all of the amino acids except for an amino acid that remained at a point of sampling (in this case, isoleucine) were taken up directly into the intracellular amino acid pools and not metabolized. That is, it was assumed that those amino acids directly became components of proteins. For the remaining amino acid, the uptake rate was calculated from the experiment and more amino acids were taken up by the cells than-incorporated into proteins. Therefore, this fact was incorporated into the model on the assumption that the excess was decomposed by metabolism. Then, the decomposition rate was calculated from the cell uptake rate and thus identified.

During the initial stage of the cultivation for about 12 hours, unlabeled amino acids derived from the medium were taken up, and these were mixed in intracellular pools of amino acids produced by a bacterium thorough metabolism of glucose as a substrate. Since cellular proteins are constituted by using these pools, they contain unlabeled amino acids. When the first sample was obtained, unlabeled amino acids contained in the medium had already been completely consumed, that is, the uptake rate was zero. Therefore, they were not incorporated into the stoichiometric matrix and the isotopomer balance equation. Unless the exchanges of the intracellular pools are very slow, intracellular amino acids in the first sample should not contain unlabeled amino acids derived from the medium. However, they were actually contained, and it was suggested that exchange reactions always occurred between the intracellular proteins and the intracellular amino acid pools. To take this point into account, Pex, a coefficient that represents these exchange reactions, was introduced into a model for analyzing the intracellular amino acid analytical data. Pex is a coefficient that represents the proportions of amino acids that return from cellular proteins to intracellular amino acid pools. The same proportion was assumed for all the amino acids except for lysine.

Since MDV of protein-hydrolyzed amino acids represents all amino acids that are incorporated into proteins from the start of the cultivation, the proportion of medium-derived amino acids is higher than that of those among intracellular amino acids. When it is assumed that the concentration of intracellular amino acids is much lower than the total protein amount, it may be considered that medium-derived unlabeled amino acids consumed during the initial stage of the cultivation were all incorporated into cellular proteins.

(4) Optimization of Metabolic Flux

A program was constructed in which MDV was calculated by using the isotopomer balance equation with free fluxes and exchange reaction fluxes as input values, and the previously inputted values of free fluxes and exchange reaction fluxes were optimized by the evolutionary algorithm (Stephani et al, Journal of theoretical Biology, 199, pp. 45-61, 1999) so that the sum of squares of the difference from the MDV obtained by the experiment should be minimized. The variables to be optimized were fluxes of ICL, malic enzyme, pentose phosphate pathway (G6PDH), values of 6 exchange reactions and Pex, which represents exchange reactions of proteins and intracellular amino acid pools. The bacterial cell yield and lysine yield were set so that 20% deviation from the input values should be accepted in order to take measurement errors in the experiment into account. The protein-hydrolyzed amino acid data and the intracellular amino acid data were separately analyzed.

To reduce the computation time, some modifications were made in a general evolutionary algorithm. Since 50,000 elements and 200 generations were found to be optimal to search the minimum value in the space of solution as a result of various examinations, these set values were used for analyses.

(5) Sensitivity Analysis

The confidence interval of free flux depends not only on variance of measured values, but also on the Jacobian matrix. The Jacobian matrix shows degree of how easily each IDV changes when the free flux changes near the optimal value. The variance of measured values for amino acids was obtained from values obtained from 3 analyses. On the basis of these values, a sensitivity matrix was calculated according to the method of Wiechert et al.

Before performing the cultivation experiment, sensitivity of the analysis model was analyzed to find the optimal mixing ratio of labeled glucose. When calculation was performed by limiting the labeled glucose to be used to 1-¹³C-Glc and U-¹³C-Glc, a mixing ratio of 50:50 in terms of percentage was found to be optimal as a result. In this experiment, a mixing ratio of 80:20, which can provide sufficient information, was adopted in view of the cost.

(6) Cultivation Experiment

Cells of WYK050/pCAB1 strain were streaked on the LB agar medium, and were cultured as stationary culture at 37° C. for 24 hours. Cells from two of the stationary culture plates were inoculated into the initial medium. The components of the medium were as described above. For the cultivation, a 1-L jar fermenter was used, and a mixture of 1-¹³C-Glc and U-¹³C-Glc at a ratio of 80:20 was used as substrates. The mixing ratio was determined by the sensitivity analysis performed beforehand. The initial liquid volume of the culture was 300 ml, and the temperature and pH were regulated to be 37° C. and 6.7, respectively. Ammonia gas was used to regulate pH. Aeration was controlled at 300 ml/min. The stirring rate was suitably regulated so that the dissolved oxygen concentration of the culture broth should be always maintained at 5% or higher. Feeding of a glucose solution was started at 17 hours after the start of the cultivation. This was immediately before the initial glucose was completely consumed. The feeding rate was suitably regulated so that the concentration of the remaining sugar in the medium should be 5 g/L or lower. A fermentation sample was obtained at 17 hours after the start of the cultivation, which was in the growth phase, and at 26 hours, which was in the stationary phase. From each sample, intracellular metabolites were extracted by the silicon oil method. Further, cells for measuring protein-hydrolyzed amino acids were also obtained at the same timings. Measurement was performed by using LC-MS and CE-MS.

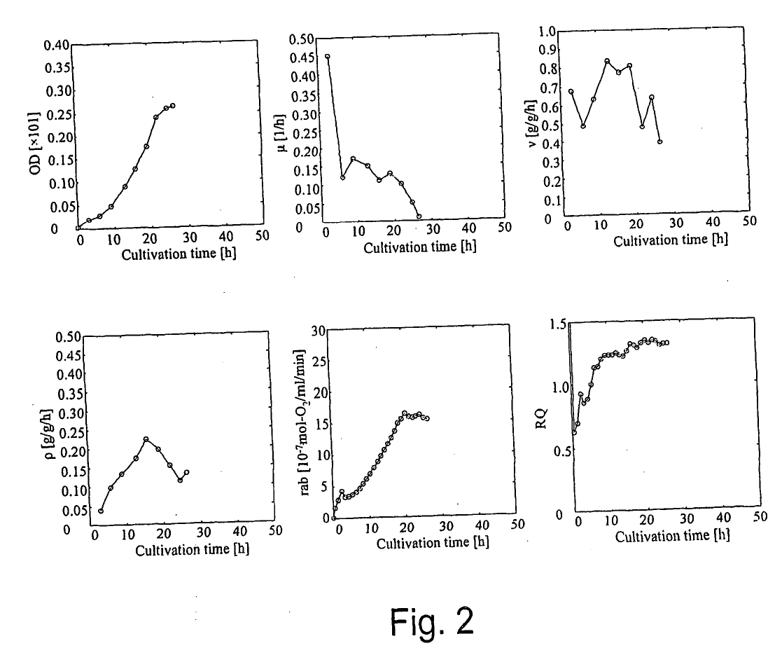

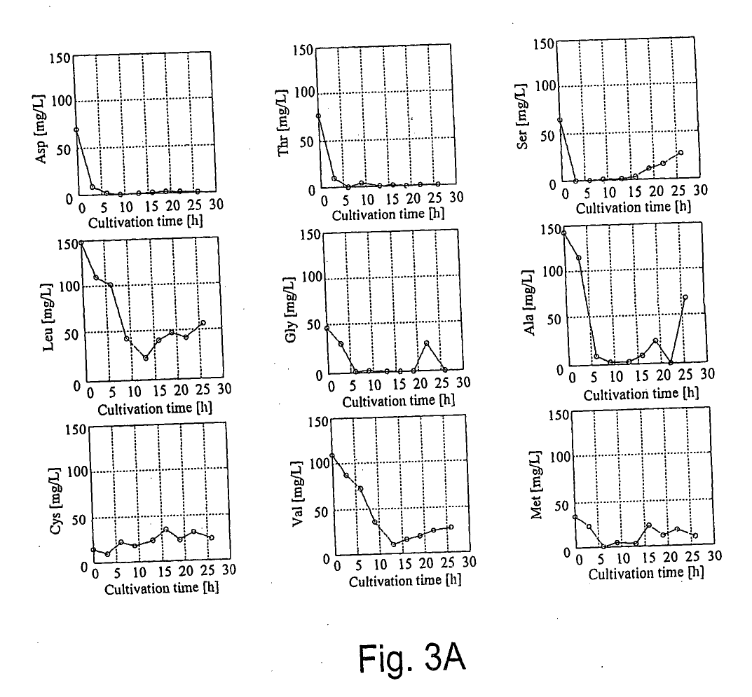

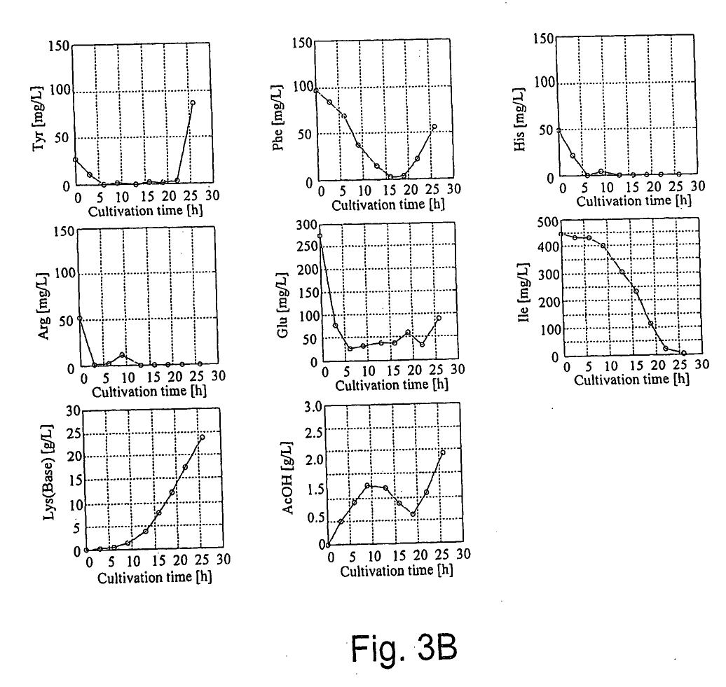

The absorption (OD), specific growth rate μ, specific sugar consumption rate ν, specific lysine production rate ρ, oxygen absorption rate rab and respiratory quotient RQ of the cells after the cultivation are shown in FIG. 2. Changes in amino acid concentrations in the medium with time are shown in FIGS. 3A and 3B. Amino acids derived from yeast extract were almost completely consumed within 15 hours after the start of cultivation. Isoleucine was completely consumed after around 20 hours from the start of the cultivation. Then, the rise of the oxygen absorption rate rab stopped, and μ and ν also decreased. To clarify the difference between the metabolic flux distributions before and after this stage, that is, in the growth phase and the stationary phase, metabolic flux analysis was performed.

The final fermentation results are shown in Table 3.

TABLE 3
Fermentation results (lysine concentration was
represented in terms of lysine hydrochloride)

| | | |
|---|---|---|
| Lysine yield | [%] | 31.1 |
| Productivity | [g/L/h] | 1.14 |
| Bacterial cell yield | [%] | 15.1 |
| Yield except for bacterial cells | [%] | 44 |
| Cultivation time | [CT] | 26.6 |
| Lysine accumulation | [g/L] | 29.8 |
| Amount of consumed sugar | [g] | 30.3 |
| Amount of obtained lysine | [g] | 9.4 |
| Amount of bacterial cells | [g] | 4.6 |

(7) Metabolic Flux Analysis
[Metabolic Flux Analysis using Protein-Hydrolyzed Amino Acid Data]

As a result of analysis of intracellular protein-hydrolyzed amino acids by LC-MS, data of the isotope ratios in the following amino acids were obtained: glycine, alanine, serine, proline, valine, threonine, phenylalanine, tyrosine, leucine and methionine. Because the data of proline for the growth phase was less reliable compared with other analytical values, they were not used, and only the data for the stationary phase were used.

Influence of natural isotopes of elements other than carbon was corrected, and then IDV of each amino acid was calculated on the basis of the experimental results. Since cellular proteins contain amino acids biosynthesized during a period from the start of cultivation through the sampling, analytical data obtained from hydrolysis of the cellular proteins are considered to be mean values in this interval. Therefore, the following numerical values as mean values in this interval were used for the analysis of data obtained at 17 hours: yield: 0.379 g of DCW/10 mmol glucose, lysine yield: 2.82 mmol/10 mmol glucose, and acetic acid uptake rate: 0.47 mmol/10 mmol glucose. When the isotopomer balance was calculated, the ratio of isotopes having a smaller mass generally tended to be higher in MDV as the experimental result compared with MDV expected from the calculation. Considering that this was due to the influence of unlabeled amino acids derived from yeast extract as a natural nutrient source, it was decided to make a correction by the aforementioned method. The unlabeled amino acids referred to herein do not mean that they consist only of ¹²C, but they also contain ¹³C at a naturally occurring proportion.

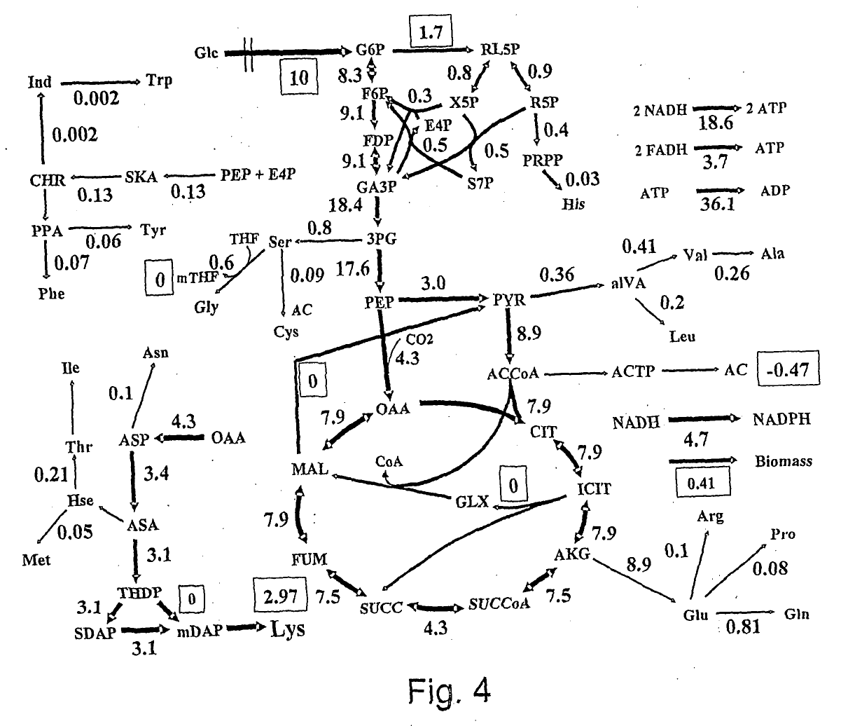

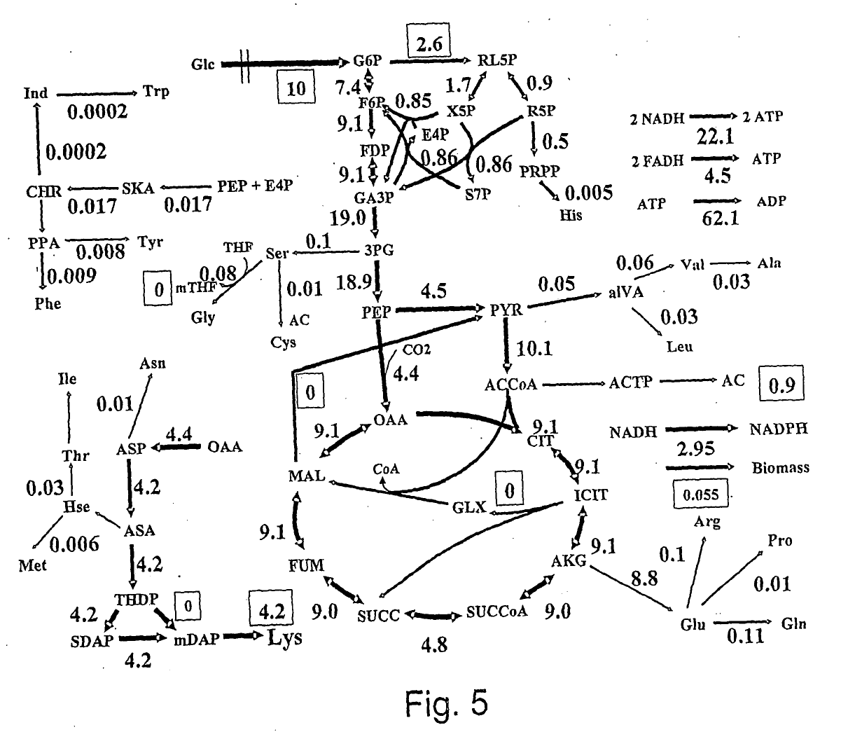

The optimization of free fluxes using the data of protein-hydrolyzed amino acids was performed several times, and substantially equivalent results were obtained. In the optimization performed with the evolutionary algorithm, calculation was performed for 50,000 elements and 200 generations. Table 4 shows MDV as the experimental result and the calculated MDV. In this calculation, the sum of errors in the results of the experiment and the calculation was 21.19. The optimized free flux values are shown in Table 5. All the metabolic flux distributions are shown in FIG. 4. The metabolic flux distributions shown in FIGS. 4 and 5 include energy metabolism reactions. The energy metabolism reactions were obtained by recalculation using stoichiometric matrices from the results calculated on the basis of transfer of carbon atoms. In summary, 16% of consumed glucose flowed into the pentose phosphate pathway, and since this flux was not sufficient to produce lysine and cells, a flux of the conversion reaction from NADH to NADPH using transhydrogenase showed a large value. Further, fluxes by ICL and malic enzyme were zero in this analysis. Reactions showing high reversibility were the reactions in the glycolysis and the pentose phosphate pathway.

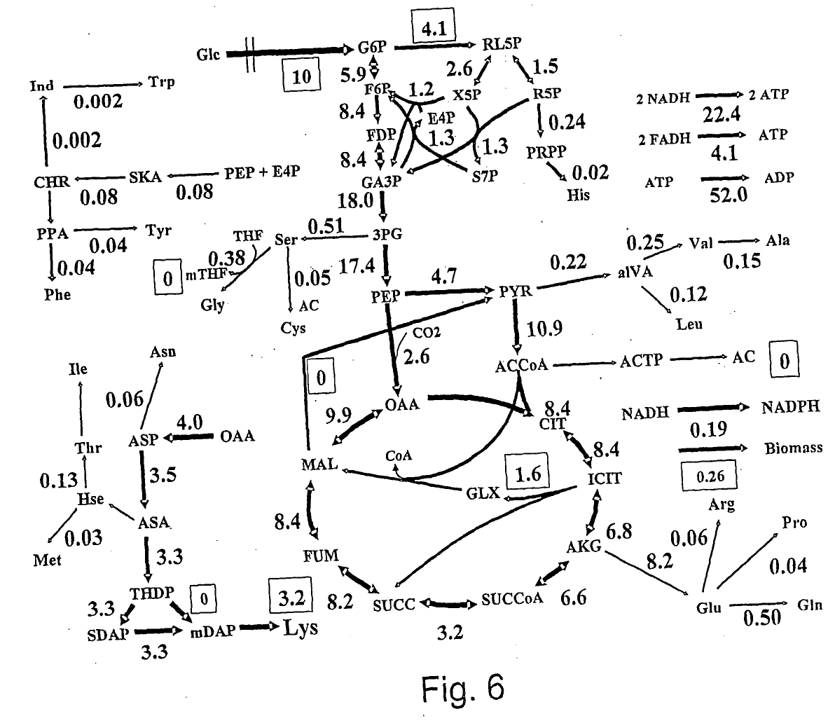

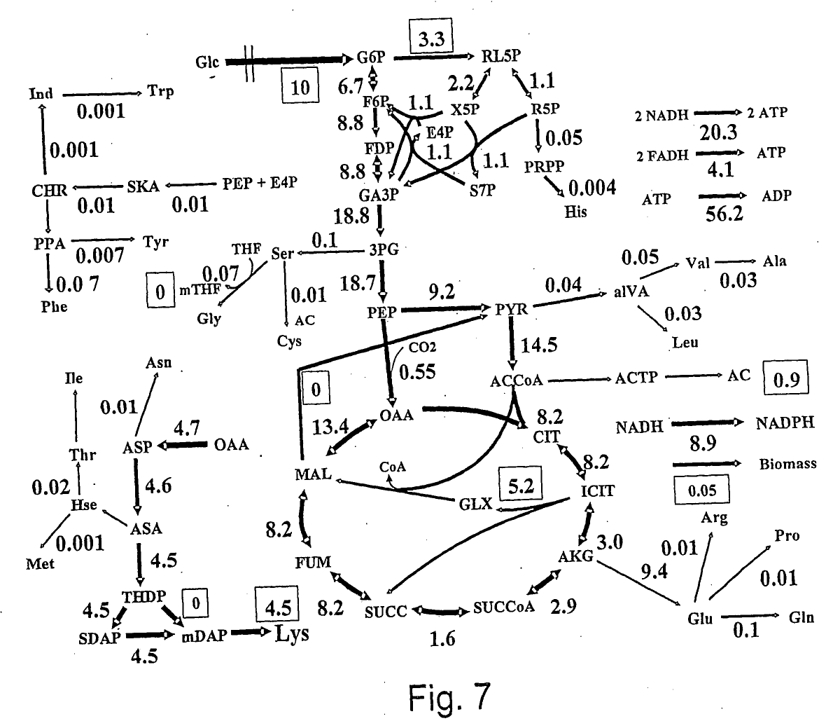

Similar analysis was performed by using the analytical data of protein-hydrolyzed amino acids obtained in the stationary phase. However, since this analytical data included information from the start of cultivation as described above, the results showed no significant difference from those obtained for the growth phase. The results are shown in FIGS. 6 and 7.

TABLE 4
Isotope distributions of amino acids (numerical
values in each column for amino acids represent molecular
weights M, M + 1, M + 2 . . . from the top)

| | Protein- | | Intracellular | | Intracellular | |
|---|---|---|---|---|---|---|
| | hydrolyzed | | amino acid | | amino acid | |
| | amino acid in | | in growth | | in stationary | |
| | growth phase | | phase | | phase | |
| | | Calcu- | | Calcu- | | Calcu- |
| Amino acid | Found | lated | Found | lated | Found | lated |
| Glycine | 0.7634 | 0.7646 | 0.7366 | 0.7363 | 0.7286 | 0.7282 |
| | 0.0801 | 0.0894 | 0.1018 | 0.1030 | 0.1070 | 0.1117 |
| | 0.1565 | 0.1460 | 0.1616 | 0.1607 | 0.1644 | 0.1601 |
| Alanine | 0.5071 | 0.6039 | 0.4534 | 0.4588 | 0.4437 | 0.4429 |
| | 0.2936 | 0.2446 | 0.3396 | 0.3326 | 0.3418 | 0.3438 |
| | 0.0476 | 0.0393 | 0.0511 | 0.0571 | 0.0651 | 0.0627 |
| | 0.1516 | 0.1123 | 0.1559 | 0.1515 | 0.1494 | 0.1506 |
| Serine | 0.4610 | 0.5072 | 0.4714 | 0.4532 | 0.4482 | 0.4364 |
| | 0.3476 | 0.3008 | 0.3418 | 0.3426 | 0.3608 | 0.3555 |
| | 0.0799 | 0.0495 | 0.0718 | 0.0593 | 0.0776 | 0.0686 |
| | 0.1115 | 0.1425 | 0.1150 | 0.1449 | 0.1134 | 0.1395 |
| Proline | 0.1308 | 0.1500 | | | 0.1352 | 0.1621 |
| | 0.3214 | 0.2939 | | | 0.3124 | 0.2813 |
| | 0.2916 | 0.3024 | | | 0.3034 | 0.2919 |
| | 0.1748 | 0.1795 | | | 0.1749 | 0.1829 |
| | 0.0662 | 0.0632 | | | 0.0601 | 0.0679 |
| | 0.0151 | 0.0110 | | | 0.0140 | 0.0139 |
| Valine | 0.5086 | 0.4622 | 0.2854 | 0.2815 | 0.2819 | 0.2539 |
| | 0.2135 | 0.2236 | 0.2829 | 0.2934 | 0.2909 | 0.3010 |
| | 0.1289 | 0.1469 | 0.2008 | 0.2019 | 0.2014 | 0.2145 |
| | 0.0942 | 0.1047 | 0.1417 | 0.1429 | 0.1452 | 0.1470 |
| | 0.0364 | 0.0430 | 0.0615 | 0.0549 | 0.0520 | 0.0581 |
| | 0.0184 | 0.0196 | 0.0277 | 0.0254 | 0.0286 | 0.0255 |
| Threonine | | | 0.3387 | 0.3702 | 0.2825 | 0.2584 |
| | | | 0.3074 | 0.2924 | 0.3218 | 0.3344 |
| | | | 0.2122 | 0.1989 | 0.2330 | 0.2413 |
| | | | 0.1048 | 0.1059 | 0.1153 | 0.1269 |
| | | | 0.0369 | 0.0327 | 0.0473 | 0.0390 |
| Asparagine | | | 0.2557 | 0.2472 | 0.2383 | 0.2410 |
| | | | 0.3435 | 0.3452 | 0.3342 | 0.3436 |
| | | | 0.2215 | 0.2408 | 0.2383 | 0.2463 |
| | | | 0.1351 | 0.1282 | 0.1427 | 0.1310 |
| | | | 0.0442 | 0.0386 | 0.0465 | 0.0381 |
| Glutamic | | | 0.1511 | 0.1574 | 0.1339 | 0.1419 |
| acid | | | 0.2855 | 0.2843 | 0.2772 | 0.2810 |
| | | | 0.2863 | 0.2930 | 0.2938 | 0.2925 |
| | | | 0.1891 | 0.1836 | 0.2013 | 0.1898 |
| | | | 0.0726 | 0.0678 | 0.0779 | 0.0798 |
| | | | 0.0154 | 0.0139 | 0.0159 | 0.0150 |
| Glutamine | | | 0.1548 | 0.1574 | 0.1374 | 0.1419 |
| | | | 0.2673 | 0.2843 | 0.2673 | 0.2810 |
| | | | 0.2971 | 0.2930 | 0.3003 | 0.2925 |
| | | | 0.1906 | 0.1836 | 0.1966 | 0.1898 |
| | | | 0.0714 | 0.0678 | 0.0798 | 0.0798 |
| | | | 0.0188 | 0.0139 | 0.0186 | 0.0150 |
| Lysine | | | 0.1396 | 0.1219 | 0.1202 | 0.1156 |
| | | | 0.2619 | 0.2605 | 0.2442 | 0.2560 |
| | | | 0.2768 | 0.2699 | 0.2586 | 0.2737 |
| | | | 0.1731 | 0.1967 | 0.2154 | 0.2007 |
| | | | 0.0957 | 0.1031 | 0.0998 | 0.1058 |
| | | | 0.0435 | 0.0388 | 0.0450 | 0.0389 |
| | | | 0.0094 | 0.0091 | 0.0168 | 0.0093 |
| Phenylalanine | 0.4345 | 0.3737 | 0.1628 | 0.1770 | 0.1193 | 0.1435 |
| | 0.1621 | 0.1738 | 0.1952 | 0.2005 | 0.1986 | 0.1991 |
| | 0.1282 | 0.1355 | 0.1932 | 0.1895 | 0.1994 | 0.1985 |
| | 0.1081 | 0.1136 | 0.1665 | 0.1634 | 0.1816 | 0.1738 |
| | 0.0781 | 0.0871 | 0.1255 | 0.1223 | 0.1356 | 0.1304 |
| | 0.0513 | 0.0607 | 0.0850 | 0.0800 | 0.0871 | 0.0840 |
| | 0.0252 | 0.0313 | 0.0424 | 0.0405 | 0.0454 | 0.0427 |
| | 0.0121 | 0.0160 | 0.0177 | 0.0189 | 0.0212 | 0.0191 |
| | 0.0049 | 0.0062 | 0.0074 | 0.0070 | 0.0086 | 0.0068 |
| | 0.0016 | 0.0021 | 0.0043 | 0.0021 | 0.0034 | 0.0021 |
| Tyrosine | 0.2435 | 0.2047 | 0.1497 | 0.1369 | 0.1135 | 0.1192 |
| | 0.1944 | 0.1976 | 0.1996 | 0.2073 | 0.2038 | 0.2032 |
| | 0.1660 | 0.1767 | 0.1967 | 0.1975 | 0.1989 | 0.2040 |
| | 0.1402 | 0.1507 | 0.1652 | 0.1701 | 0.1720 | 0.1770 |
| | 0.1085 | 0.1158 | 0.1263 | 0.1287 | 0.1324 | 0.1345 |
| | 0.0789 | 0.0806 | 0.0861 | 0.0858 | 0.0945 | 0.0881 |
| | 0.0386 | 0.0416 | 0.0439 | 0.0433 | 0.0485 | 0.0445 |
| | 0.0198 | 0.0213 | 0.0212 | 0.0205 | 0.0238 | 0.0200 |
| | 0.0080 | 0.0083 | 0.0082 | 0.0076 | 0.0095 | 0.0074 |
| | 0.0022 | 0.0028 | 0.0031 | 0.0023 | 0.0031 | 0.0021 |
| Leucine | 0.4232 | 0.3247 | | | | |
| | 0.1676 | 0.1937 | | | | |
| | 0.1740 | 0.2167 | | | | |
| | 0.1343 | 0.1573 | | | | |
| | 0.0733 | 0.0794 | | | | |
| | 0.0229 | 0.0239 | | | | |
| | 0.0048 | 0.0043 | | | | |
| Methionine | 0.2816 | 0.3200 | | | | |
| | 0.2705 | 0.2436 | | | | |
| | 0.2298 | 0.2249 | | | | |
| | 0.1392 | 0.1376 | | | | |
| | 0.0632 | 0.0609 | | | | |
| | 0.0156 | 0.0130 | | | | |

TABLE 5
Values of free fluxes, exchange coefficients and protein
degradation coefficients optimized by optimization algorithm

| | Protein- | | Intracellular |
|---|---|---|---|
| | hydrolyzed | Intracellular | amino acid in |
| | amino acid in | amino acid in | stationary |
| | growth phase | growth phase | phase |
| Free flux | | | |
| G6PDH | 1.65 | 4.15 | 3.32 |
| ICL | 0 | 1.62 | 5.23 |
| Malic enzyme | 0 | 0.164 | 0 |
| Exchange | | | |
| coefficient | | | |
| G6PDH | 0.174 | 0.152 | 0.179 |
| Glycolysis | 0.434 | 0.699 | 0.752 |
| PEPC | 0 | 0.096 | 0.158 |
| ICD | 0 | 0 | 0 |
| TCA cycle | 0 | 0.408 | 0.054 |
| Malic enzyme | 0 | 0 | 0 |
| Protein | | 0.210 | 0.269 |
| degradation | | | |
| coefficient | | | |

[Metabolic Flux Analysis using Analytical Data of Intracellular Amino Acids]

Intracellular amino acids were analyzed by LC-MS to obtain MDV of the following amino acids: glycine, alanine, serine, proline, valine, threonine, asparagine, glutamine, glutamic acid, lysine, phenylalanine and tyrosine. As the input data into the analysis model, the following data obtained at the point of sampling were used: yield: 0.272 g of DCW/10 mmol glucose, lysine yield: 3.30 mmol/10 mmol glucose and acetic acid uptake rate: 0 mmol/10 mmol glucose. Since the intracellular analytical data was the data as of the sampling, it was significantly different from the aforementioned protein hydrolysis data representing mean values.

When MDV of each amino acid was estimated by using the isotopomer balance equation, the proportion of isotopes having a smaller mass also tended to become higher than in MDV obtained from the actual experimental results also in this case, although the difference was smaller than that observed when the data of protein-hydrolyzed amino acids were used. When the sample was obtained, medium-derived unlabeled amino acids had already been completely consumed, and it was suggested that the experimental results were influenced by those. Therefore, to take the influence into account, the algorithm was changed to optimize numerical values by defining Pex, a ratio of exchange reactions with intracellular amino acid pools through degradation of cellular proteins. Pex was assumed to be equal for all amino acids.

On the basis of the above, optimization was performed with the evolutionary algorithm. As a result, the sum of errors in MDV obtained by the experiment and MDV obtained by the calculation was minimized, i.e., 7.57. Also in this case, 50,000 elements and 200 generations were used in the evolutionary algorithm. MDV obtained by the experiment and MDV obtained by the calculation are shown in Table 5. The optimized free flux values are shown in Table 5. All the metabolic flux distributions are shown in FIG. 6. The significant difference from the results of the analysis using protein-hydrolyzed amino acid data was that fluxes of ICL and G6PDH were large.

Similarly, analysis was performed by using the intracellular amino acid data obtained for the stationary-phase. As a result, the sum of errors in MDV obtained by the experiment and MDV obtained by the calculation was minimized, i.e., 8.71. As the input data into the analysis model, the following data obtained at the point of sampling were used: bacterial cell yield: 0.055 g of DCW/10 mmol glucose, lysine yield: 4.27 mmol/10 mmol glucose and acetic acid discharge rate: 0.9 mmol/10 mmol glucose.

The significant difference of the stationary phase compared with the growth phase is that the ICL flux increased by nearly 3 times in the growth phase, and the PEPC flux became zero.

### INDUSTRIAL APPLICABILITY

The present invention provides a metabolic flux analysis method, which uses isotope-labeled compounds and shows little analytical errors.

1. A method for analyzing an intracellular metabolic flux comprising determining the intracellular metabolic flux from analytical values of cells cultured in a medium containing an isotope-labeled substrate as a carbon source on the basis of an intracellular metabolic flux model constructed for the intracellular metabolic flux to be analyzed,
which satisfies at least one of the following conditions (a) to (c):
(a) the analytical values of cells include an analytical value of isotope distribution in an intracellular metabolite included in the intracellular metabolic flux model, and the analytical value of isotope distribution in the intracellular metabolite is corrected for a degree of synthesis and degradation between the intracellular metabolite and a cell component produced by integration of the intracellular metabolite;
(b) the intracellular metabolic flux model includes at least one of useful compounds and major metabolic intermediates thereof; the analytical values of cells include an uptake rate of a compound in a medium into cells, said compound being identical to the intracellular metabolite and unlabeled with an isotope, and an analytical values of isotope distribution in at least one of the useful compounds and the major metabolic intermediates thereof; and the analytical value of isotope distribution in at least one of the useful compounds and the major metabolic intermediates thereof is corrected for influence of a rate of inflow into a metabolic pathway on the isotope distribution in at least one of the useful compounds and the major metabolic intermediates thereof on the assumption that a rate obtained by subtracting a rate of integration into a cell component from the uptake rate is the rate of inflow into the metabolic pathway;
(c) the intracellular metabolic flux model includes a carbon dioxide fixation reaction and a carbon dioxide production reaction, and carbon dioxide used in the fixation reaction is assumed as carbon dioxide produced in the production reaction.

2. The method according to claim 1, which satisfies the condition (a), and wherein the analytical value of the isotope distribution in the intracellular metabolite is corrected by constructing the intracellular metabolic flux model to include an exchange reaction of the intracellular metabolite and the cell component produced by integration of the intracellular metabolite, and using the analytical values of cells including the analytical value of the isotope distribution in the intracellular metabolite and an analytical value of isotope distribution in a degradation product of the cell component.

3. The method according to claim 1, which satisfies the condition (a), and wherein the analytical value of isotope distribution in the intracellular metabolite is corrected by 1 ) the step of measuring isotope distribution in the intracellular metabolite and isotope distribution in a degradation product of the cell component, and 2) the step of optimizing the degree of synthesis and degradation between the intracellular metabolite and the cell component on the basis of the results obtained in the step 1), by an optimization algorithm.

4. The method according to claim 3, wherein the degree of synthesis and degradation is expressed as a variable defined by an exchange reaction coefficient.

5. The method according to claim 3, wherein the optimization algorithm is an evolutionary algorithm.

6. The method according to claim 3, wherein the intracellular metabolite is at least one of an amino acid and an organic acid, and the cell component is a protein.

7. The method according to claim 2, which satisfies the condition (a), and wherein the analytical value of the isotope distribution in the degradation product of the cell component is corrected for influence of integration of a compound in the medium into the cell component, said compound being identical to the intracellular metabolite and unlabeled with an isotope.

8. The method according to claim 7, wherein the compound which is unlabeled with an isotope is an amino acid.

9. The method according to claim 1, which satisfies the condition (b), and wherein the compound which is unlabeled with an isotope is an amino acid.

10. The method according to claim 9, wherein the amino acid is isoleucine.

11. The method according to claim 1, wherein the cells are those of a microorganism having an ability to produce a useful compound.

12. The method according to claim 11, wherein the useful compound is at least one of an amino acid and an organic acid.

13. The method according to claim 1, wherein culture of the cells is batch culture or fed-batch culture.

14. The method according to claim 1, wherein the intracellular metabolite is at least one of an amino acid and an organic acid, or a major metabolic intermediate thereof, or both.

15. The method according to claim 1, wherein the isotope distribution is measured by mass spectrometry.

16. A program for causing a computer to function as a means for storing an intracellular metabolic flux model constructed for an intracellular metabolic flux to be analyzed, a means for inputting analytical values of cells cultured in a medium containing isotope-labeled substrates as a carbon source, a means for determining a variable of the intracellular metabolic flux model on the basis of the intracellular metabolic flux model and the analytical values of cells to determine the intracellular metabolic flux, and a means for outputting the determined intracellular metabolic flux, wherein the intracellular metabolic flux model is constructed, or the variable of the intracellular metabolic flux model is calculated, or both, so that at least one of the following conditions (a) to (c) is satisfied:
(a) the analytical values of cells include an analytical value of isotope distribution in an intracellular metabolite included in the intracellular metabolic flux model, and the analytical value of isotope distribution in the intracellular metabolite is corrected for a degree of synthesis and degradation between the intracellular metabolite and a cell component produced by integration of the intracellular metabolite;
(b) the intracellular metabolic flux model includes at least one of useful compounds and major metabolic intermediates thereof; the analytical values of cells include an uptake rate of a compound in a medium into cells, said compound being identical to the intracellular metabolite and unlabeled with an isotope, and an analytical value of isotope distribution in at least one of the useful compounds and the major metabolic intermediates thereof; and the analytical value of isotope distribution in at least one of the useful compounds and the major metabolic intermediates thereof is corrected for influence of a rate of inflow into a metabolic pathway on the isotope distribution in at least one of the useful compounds and the major metabolic intermediates thereof on the assumption that a rate obtained by subtracting a rate of integration into a cell component from the uptake rate should be the rate of inflow into the metabolic pathway;
(c) the intracellular metabolic flux model includes a carbon dioxide fixation reaction and a carbon dioxide production reaction, and carbon dioxide used in the fixation reaction is assumed as carbon dioxide produced in the production reaction.

17. A computer-readable recording medium, which records the program as defined in claim 16.
